(19) United States
(12) Patent Application Publication
(10) Pub. No.: US 2017/0281094 A1
(43) Pub. Date: Oct. 5, 2017

(54) Information Based Machine Learning Approach to Elasticity Imaging

(71) Applicant: The Board of Trustees of the University of Illinois, Urbana, IL (US)

(72) Inventors: Jamshid Ghaboussi, Urbana, IL (US); Cameron Lee Hoerig, Champaign, IL (US); Michael F Insana, Urbana, IL (US)

(73) Assignee: The Board of Trustees of the University of Illinois, Urbana, IL (US)

(21) Appl. No.: 15/480,270

(22) Filed: Apr. 5, 2017

Related U.S. Application Data
(60) Provisional application No. 62/318,297, filed on Apr. 5, 2016.

(51) Int. Cl. A61B 5/00; A61B 8/14; A61B 8/00

(52) U.S. Cl. CPC A61B 5/7264; A61B 5/0048; A61B 8/4483; A61B 8/14; A61B 8/4444; A61B 8/587

(57) ABSTRACT

Systems and methods are provided for employing informational models trained using the Autoprogressive Algorithm to learn the mechanical behavior of biological materials using a sparse sampling of force and displacement measurements. The constitutive matrix normally used to solve the inverse problem is replaced with ANNs.

### CROSS REFERENCE TO RELATED APPLICATIONS

This application claims the benefit of the filing date of U.S. provisional patent application No. 62/318,297, entitled “Information Based Machine Learning Approach to Elasticity Imaging”, which was filed on Apr. 5, 2016, by the same patentee of this application and which shares the same inventor(s) as this application. That provisional application is hereby incorporated by reference as if fully set forth herein.

### STATEMENT REGARDING FEDERALLY SPONSORED RESEARCH OR DEVELOPMENT

This technology was made with government support under CA168575 awarded by National Cancer Institute. The government has certain rights in the technology.

### FIELD OF THE TECHNOLOGY

The technology of this disclosure relates generally to mechanical property imaging of biological media, and more specifically but not exclusively to employing informational models to predict full stress and strain distributions from applied forces by employing the Autoprogressive Algorithm that uses artificial neural networks.

### BACKGROUND OF THE INVENTION

Medical elasticity imaging encompasses a broad range of techniques for imaging mechanical properties of biological tissues. Conventional approaches to biological tissue imaging begin by tracking displacements resulting from a weak mechanical stimulus applied to the tissue being imaged. The elements of a constitutive matrix are estimated by combining time varying displacement information with measurements of, or assumptions about, the associated forces into a collection of linear equations. Strong symmetry assumptions are required to create a problem that can be solved using available data and can reduce the constitutive matrix elements to just one or two variables that are mapped into images.

Elasticity imaging reveals how tissue stiffness and/or viscosity vary with position and time. These basic mechanical properties can indicate regions of inflammation, edema, hypertrophy, and fibrosis that accompany the presence of disease processes. However, simplifying assumptions, which enter conventional analysis as tissue models, are often unjustified given the structural complexity of most tissues. Violating the assumptions of a tissue model can distort the description of the mechanical environment needed for diagnostic decision making. Unjustified assumptions also limit the vast possibilities for learning more about the role of mechano-biology in revealing disease processes. Furthermore, conventional overly simple models generally discard some of the information gathered during an imaging exam.

Since the mechano-environment can promote and inhibit tumor-cell progression, elasticity imaging is being developed to measure important mammary-cell properties that influence malignant tumor progression. Several conventional approaches to elasticity imaging exist for probing the landscape of mechanical properties from complex tissue structures. Some approaches examine compressive, shear, or flow properties; others describe fluid diffusion and tissue porosity to visualize changes in morphology known to accompany malignant-cell transitions. Each conventional imaging technique probes tissues at a load frequency that engages some deformation mechanisms and ignores others. For example, small deforming forces are consistent with the linear-viscoelastic assumptions of the Kelvin-Voigt model that describes elastic and viscous responses. However, these two parameters are not necessarily the most diagnostic, they are just the most accessible.

With conventional techniques, applied forces are carefully selected to satisfy constitutive model assumptions, often without careful consideration of how the load restricts access to specific disease information. The field is just now beginning to understand how applied forces link to material and structural features to reveal information, and so basing measurement methods on constitutive models, which is the conventional method, is unlikely to be the best practice. It is more likely that conventional approaches to elasticity imaging limit its potential to improve cancer diagnosis.

It would be advantageous to provide systems and methods for elasticity imaging of biological tissue which do not require constitutive model assumptions. It would be advantageous to provide systems and methods for elasticity imaging of biological tissue which provide accurate prediction of stresses and strains throughout selected regions of the tissue at user defined resolutions. It would be advantageous to provide systems and methods for elasticity imaging of biological tissue which employ informational modelling.

### BRIEF SUMMARY OF THE TECHNOLOGY

Many advantages will be determined and are attained by the technology of the present disclosure, which in a broad sense provides systems and methods for elasticity imaging of biological tissue using informational models to predict stress and strain distributions from applied forces without being required to engage specific constitutive models.

One or more embodiments of the technology provides a computer-implemented method for estimating stress and strain distributions in biological tissue without requiring estimations of underlying constitutive models. The method includes applying with a probe, a series of forces to the tissue. The method also includes recording a series of displacement measurements respectively over a period of time resulting from the forces, such that the measurements are recorded from multiple locations on the tissue. The method includes providing the force and displacement measurements to a processor based device, and using the processor based device to generate, based at least in part on the force and displacement measurements, the estimates of stress and strain distributions in the tissue.

One or more embodiments of the technology provides a system for characterization of mechanical properties of biological tissue. The system includes a force producing device and a measuring device. The measuring device is configured to measure external force applied to the biological tissue. The system also includes a processor in electrical communication with the measuring device. The processor is programmed to measure a set of internal and external displacements arising from the external force applied to the tissue. The processor is also programmed to develop an informational model of mechanical properties of the tissue using the Autoprogressive Algorithm.

One or more embodiments of the technology provides a method for generating a nonparametric, informational model of mechanical properties of biological tissue. The method includes applying an external force with an ultrasound probe having an array transducer, to the tissue at a first position and transmitting a radio frequency (“RF”) signal into the tissue. The method further includes applying an external force with the ultrasound probe, to the tissue at another position and transmitting the RF signal into the tissue. An RF echo frame is acquired and recorded each time the RF signal is transmitted into the tissue. The method also includes recording with a force-torque transducer, a surface force generated by the probe after each compression and measuring an internal displacement using a speckle-tracking algorithm. The force and the displacement are then entered into the Autoprogressive (“AutoP”) Algorithm.

The technology will next be described in connection with certain illustrated embodiments and practices. However, it will be clear to those skilled in the art that various modifications, additions and subtractions can be made without departing from the spirit or scope of the claims.

### BRIEF DESCRIPTION OF THE DRAWINGS

For a better understanding of the technology, reference is made to the following description, taken in conjunction with the accompanying drawings, in which like reference characters refer to like parts throughout, and in which:

FIG. 1 illustrates an experimental setup of a system for performing elasticity imaging of biological tissue in accordance with one or more aspects of the described technology;

FIG. 2 illustrates a flow diagram of the Autoprogressive Algorithm in accordance with one or more aspects of the described technology;

FIG. 3 illustrates how an ANN replaces the constitutive matrix to relate stresses and strains;

FIG. 4 illustrates finite-element meshes used in the Autoprogressive Algorithm and an example of a B-mode image acquired during data acquisition in accordance with one or more aspects of the described technology;

FIG. 5 illustrates informational model estimates of stress and strain maps in accordance with one or more aspects of the described technology;

FIG. 6 illustrates a conventional FEA model estimates of stress and strain maps for the same model as that used in FIG. 5;

FIG. 7 illustrates estimates of axial stress images for complex phantom geometries in accordance with one or more aspects of the described technology;

FIG. 8 illustrates various comparisons between the predicted and measured displacements in accordance with one or more aspects of the described technology;

FIG. 9 illustrates stress responses of the Autoprogressive Algorithm trained artificial neural network to strain probes in accordance with one or more aspects of the described technology;

FIG. 10 illustrates a comparison of the axial stress responses to lateral strain probes in accordance with one or more aspects of the described technology;

FIG. 11 illustrates a comparison of the shear stress responses to shear strain probes in accordance with one or more aspects of the described technology;

FIG. 12 illustrates a comparison of an estimated shear stress field in accordance with one or more aspects of the described technology;

FIG. 13 illustrates Young's modulus images generated from trained informational models when the internal inclusion boundaries were estimated in accordance with one or more aspects of the described technology;

FIG. 14 illustrates informational models for 3-D materials using only a few internal displacements (as well as forces and displacements of the ultrasound probe) to estimate the 6-component stress and strain vectors throughout the entire phantom, from which the Young's moduli were estimated in accordance with one or more aspects of the described technology; and,

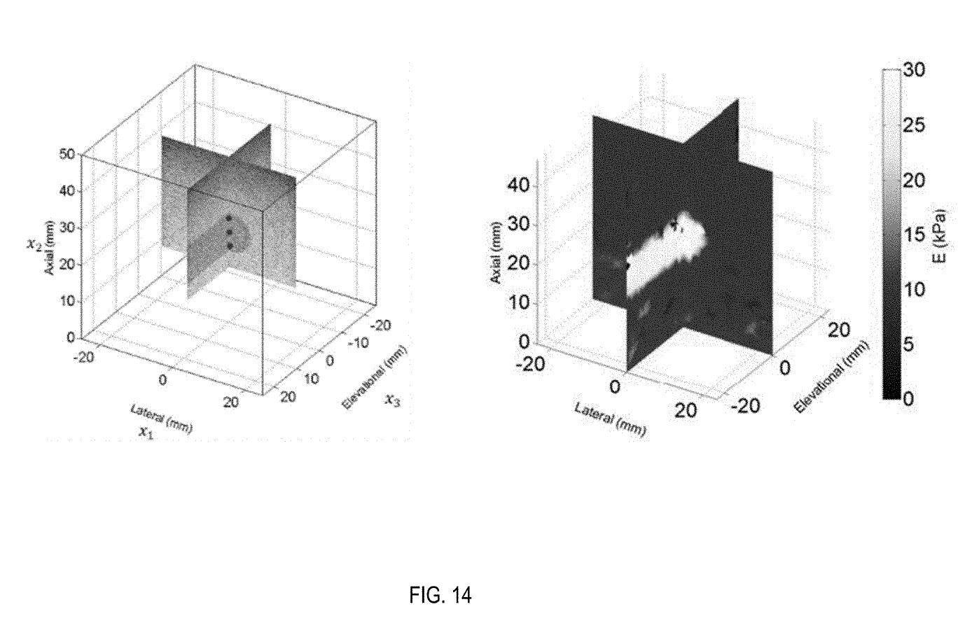

FIG. 15 illustrates one or more aspects of the described technology applied to rabbit kidneys embedded in a gelatin mixture.

The technology will next be described in connection with certain illustrated embodiments and practices. However, it will be clear to those skilled in the art that various modifications, additions, and subtractions can be made without departing from the spirit or scope of the claims.

### DETAILED DESCRIPTION OF THE TECHNOLOGY

Referring to the figures in detail wherein like reference numerals identify like elements throughout the various figures, there is illustrated systems and methods for medical elasticity imaging in biological tissue using informational models to predict stresses and strains from applied forces.

The following description is provided as an enabling teaching as it is best, currently known. To this end, those skilled in the relevant art will recognize and appreciate that many changes can be made to the various aspects described herein, while still obtaining the beneficial results of the technology disclosed. It will also be apparent that some of the desired benefits can be obtained by selecting some of the features while not utilizing others. Accordingly, those with ordinary skill in the art will recognize that many modifications and adaptations are possible, and may even be desirable in certain circumstances, and are a part of the technology described. Thus, the following description is provided as illustrative of the principles of the technology and not in limitation thereof. Discussion of an embodiment, one or more embodiments, an aspect, one or more aspects, a feature or one or more features is intended be inclusive of both the singular and the plural depending upon which provides the broadest scope without running afoul of the existing art and any such statement is in no way intended to be limiting in nature. Technology described in relation to one of these terms is not necessarily limited to use in that particular embodiment, aspect or feature and may be employed with other embodiments, aspects and/or features where appropriate.

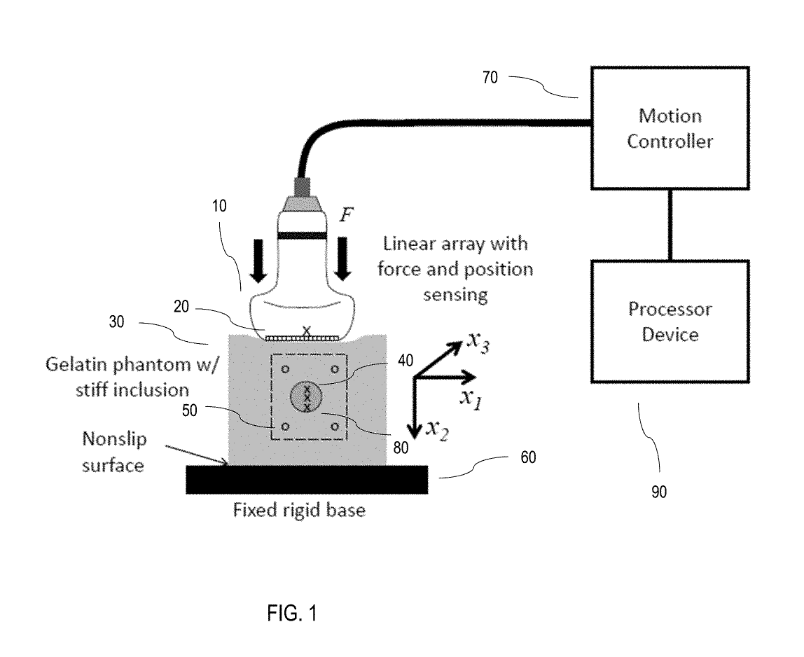

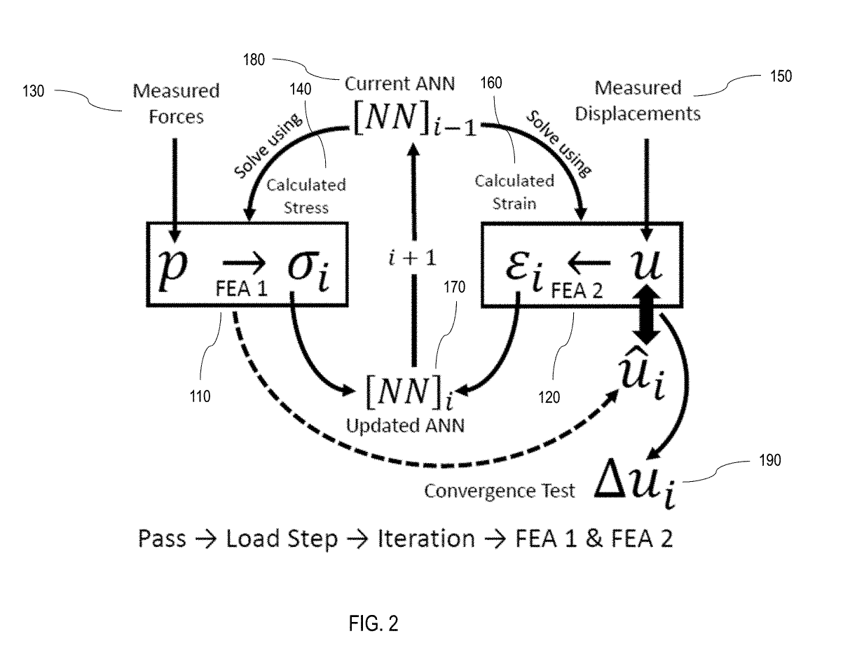

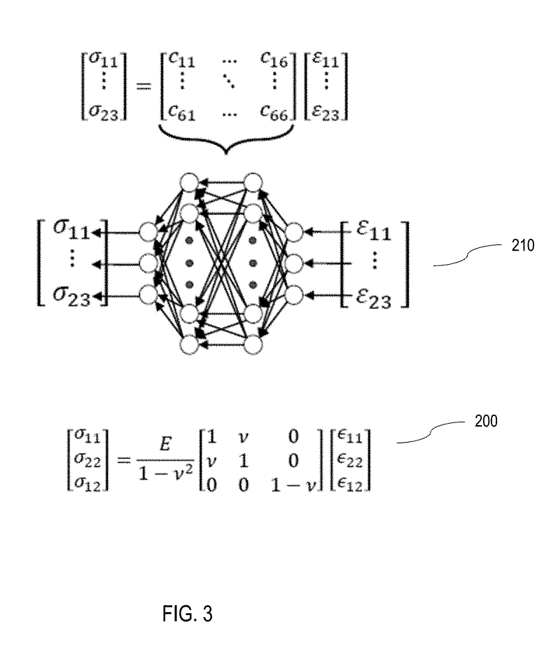

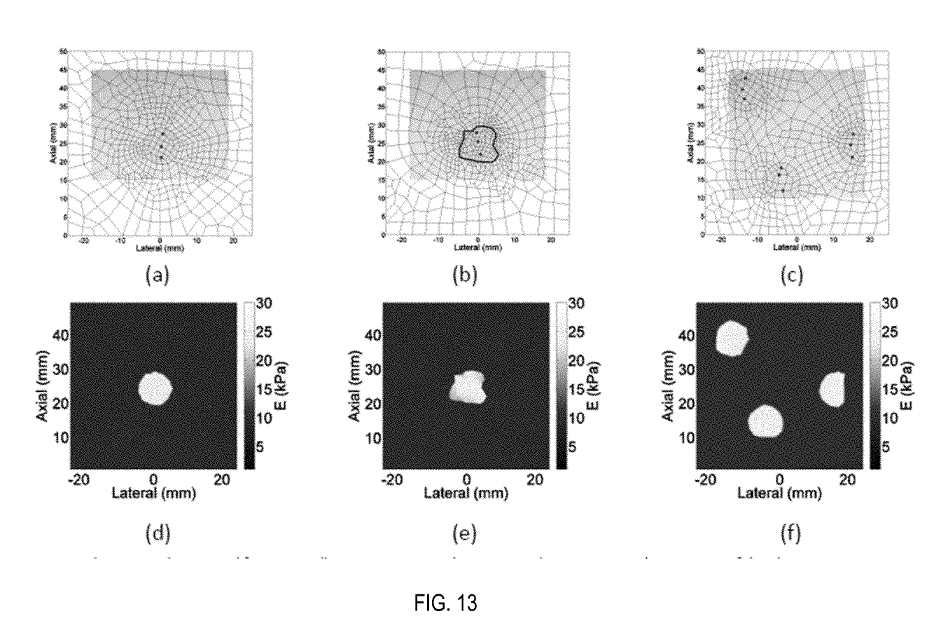

The following description provides an approach to elasticity imaging that does not require assumptions about tissue mechanical properties, including linearity and isotropy, although it does not necessarily restrict the use either. The initial focus is on quasi-static methods that generate full stress and strain maps from force-displacement measurements and knowledge of external object shape. Internal geometry can be estimated and may not actually be required. FIG. 13 illustrates 3 Young's modulus images generated from trained informational models when the internal inclusion boundaries were estimated (sometimes poorly). Estimation of constitutive-matrix elements for elasticity imaging may occur retrospectively, once the stress and strain fields have been estimated. The technique (illustrated in FIG. 1) accepts a time-series of surface force-displacement measurements made while pressing a rigid or possibly but not preferably substantially-rigid ultrasound probe 10 having a transducer 20 into the tissue surface. Those skilled in the art will recognize that the systems and methods are not limited to ultrasound. As the probe 10/transducer 20 is being pressed to the tissue 30, it records echo data which is used to estimate a time series of displacements 40, 50 within select points in the tissue 30. These force-displacement data are input into the Autoprogressive (AutoP) Algorithm (FIG. 2) (such as the one described in J. Ghaboussi, D. A. Pecknold, M. Zhang, and R. M. Haj-ali, “Autoprogressive Training of Neural Network Constitutive Models,” International Journal for Numerical Methods in Engineering, vol. 42, no. November 1996, pp. 105-126, 1998 and U.S. Pat. No. 7,447,614 entitled “Methods and Systems for Modeling Material Behavior” which are both hereby incorporated by reference as if fully set forth), that employs two finite-element analyses (FEAs) 110/120 to exploit (a) equilibrium conditions relating forces 130 and stresses 140 and (b) compatibility requirements relating displacements 150 and strains 160. These FEA processes 110/120 occur simultaneously and iteratively. Those skilled in the art will recognize that the processes are not required to occur simultaneously. Those skilled in the art will also recognize that the Autoprogressive Algorithm may be realized using a processing device such as a computer, an application specific integrated circuit, or any other sufficiently programmed processing device (not illustrated). The above can be demonstrated by relating the FEA processes 110/120 to each other through one or more artificial neural networks (ANNs) 170, 180 that learn and record material properties. The FEAs 110, 120 are independent of each other in that the solution of one does not affect the solution of the other. However, the solutions from each are dependent on the state of the ANN material models 170, 180. When the ANNs 170, 180 converge to the true material properties, the solutions from both FEA 1 110 and FEA 2 120 should match. The ANNs 170, 180 take the place of the conventional constitutive matrix, removing the need for simplifying assumptions (e.g. the need for assumptions about material properties and boundary values) (FIG. 3). The ANN 170, 180 learns material properties by transforming force-displacement data 130, 150 into stress-strain data 140, 160 through FEAs 110, 120. The result of AutoP training is a comprehensive, nonparametric, informational model of the tissue mechanical properties. Subsequently, without the need for further patient measurements, the informational model can be probed to find imaging parameters in the range of mechanical stimuli that were used to train the model. In quasi-static deformation analysis, a mechanical stimulus applied to the medium may be sensed at every contiguous location in that medium. Consequently, a few well-positioned measurements made during deformation results in a complete mechanical description, even for large deformations in nonlinear media. The material properties learned through training with AutoP are stored in the distributed ANN connection weights.

FIG. 1 illustrates an exemplary system in accordance with one or more features of the disclosed technology being used on a gelatin phantom 30 which represents tissue. Relatively simple physical phantoms with known properties are employed to demonstrate the method. Heterogeneous phantoms composed of linear-elastic gelatin gels with known shapes and stiffness were built to develop and validate these methods and thus the following examples will provide one of ordinary skill in the art with an understanding of the technology. An ultrasonic linear array is used to compress phantoms under plane-stress conditions. Those skilled in the art will recognize that it can be modeled as plane-stress given the geometry of the phantoms. Plane-stress is only a 2D approximation of the 3D problem, though. Thus, it is possible and maybe even probable that a configuration which employs a spatial-stress or multiple plane-stress conditions may be employed. The three stress and three strain fields for this 2-D measurement geometry, along with the spatially varying Young's modulus, can be accurately reconstructed even for locations outside of the imaged region. Those skilled in the art will recognize that the following examples are equally applicable to use with actual tissue.

In an experimental configuration, a centrally-positioned ultrasound probe 10 connected to a force/torque transducer 20 (which may be directly connected to the probe 10 or connected via other equipment such as a computer) is maneuvered by a conventional positioning system (preferably a 3-D positioning system) (not illustrated). A gelatin phantom 30 placed on a fixed non-slip base 60 is compressed from above with surface force p as a radio frequency (RF) echo frame (dashed line region) is acquired. Speckle-tracking applied to echo signals measures displacements within the RF echo frame. Locations marked ‘x’ 40 indicate positions where displacements were applied in FEA 2 120 for training. These displacements and others throughout the frame marked ‘o’ 50 are applied for convergence testing. Those skilled in the art will recognize that this configuration is a design choice and is only a possible configuration. A different arrangement of locations for displacement data may be used (i.e., the locations of the ‘x’ and ‘o’ are variable) and other configurations which do not employ ultrasound imaging and/or the data acquisition setup may be employed without departing from a scope of one or more claims. For example, free-hand scanning of tissues may be employed. Such a setup will likely implement an external vision system and/or inertial detectors attached to a scanning device to track its location in space. The scanning device would also have a way to measure forces and may (but not necessarily) allow a way to measure internal displacements. It may also be possible to “image without an imaging device”.

A constitutive model relates a mechanical system's output stress to input strain (or vice-versa) through mathematical equations based on known physical principles. For systems too complex to have a complete reductionist description, informational models can describe input-output relationships via machine learning techniques and training involving repeated exposure to high-quality experimental data.

Force-Displacement Measurements: The basic measurement technique involves pressing an ultrasound probe 10 having a linear-array transducer 20 into a gelatin phantom 30 in a series of 15-30 discrete steps (FIG. 1) to deliver a 3% applied strain. Those skilled in the art will recognize that the number of steps may be modified and while 3% is employed to ensure that the system stays within the linear-elastic regime for the gelatin materials, other known percentages may be employed without departing from a scope or spirit of one or more claims. Probe 10 position is controlled with submillimeter positional accuracy using a motion controller. The surface force generated by the ultrasound probe 10 after each stepped compression is recorded using a 6-axis force-torque transducer 20 (e.g. ATI Industrial Automation, Apex, N.C.) in a rig that couples the ultrasound probe 10 to the motion controller 70. Those skilled in the art will recognize that this is a design choice. A downward compressive force P is applied along the axis of the ultrasound beam (x₂ axis) (although those skilled in the art will recognize that the system is not so limited). After each compressive step, an RF echo-signal frame is recorded so that a set of internal displacements can be measured using a speckle-tracking algorithm. Those skilled in the art will recognize that other methods may be employed for measuring and/or estimating internal displacements without departing from a scope of one or more claims. As illustrated in FIG. 2, force-displacement measurements made following each compression step are introduced into AutoP to develop a model.

Phantoms Measurements: Each phantom 30 tested is a 50×50×50 mm³ cube of material that embeds one or three stiff cylindrical inclusions 80, 10 mm in diameter and 50 mm long. The long axis of each inclusion 80 is oriented along the x₃ axis (into plane of FIG. 1). Each phantom component is a linear-elastic, nearly incompressible material with a gelatin concentration that determines stiffness. Macro-indentation methods are applied to independently estimate the Young's modulus. These values are summarized in Tables I and 2 as ‘indentation meas.’

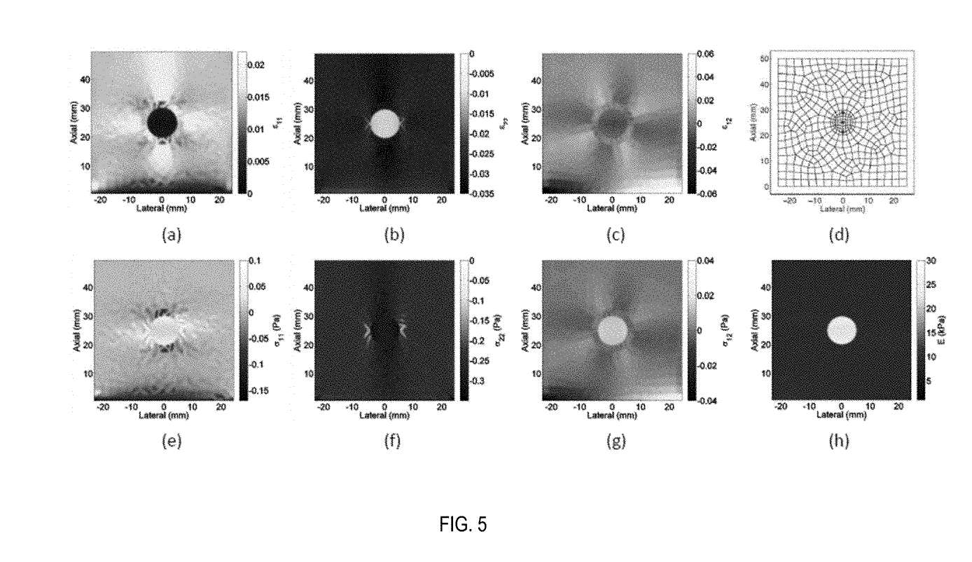

TABLE 2
Young's modulus values and displacement errors for the four phantoms studied. Estimated values were
calculated from modulus images. Indentation measurements were made on samples for comparison.
Cases refer to training scenarios described in Table 1. For Phantoms 1-3, rotation angles refer to the
orientation of the phantoms illustrated in FIG. 5. Rotation angle for Phantom 4 refers to the rotation of the
US probe about the x₂ axis away from the (x₁, x₂) plane. Displacement errors are the mean
of c_{μ} (Eq. 4) computed from FRA_{ADD}¹⁰ over all ten load increments for displacements at
all nodes in the imaged region.

| Young's Modulus Measurements of Phantom Components | | | | | |
|---|---|---|---|---|---|
| | ANN Estimated (kPa) | | Indentation Meas. (kPa) | | Displacement Errors |
| {Phan. #} (Case) [Rot. Angle] | Background | Inclusion | Back. | Inc. | c_{μ}/cₘₐₓ |

2-D Models, Known Geometry

| | | | | | |
|---|---|---|---|---|---|
| {1} (a) [0 deg] | 8.01 ± .5599 | 19.35 ± .2022 | 9.16 | 22.9 | 0.17 ± 0.13/0.58 |
| {1} (b) [0 deg] | 11.01 ± .1189 | 27.71 ± .5141 | | | 0.12 ± 0.10/0.48 |
| {1} (c) [0 deg] | 10.18 ± .3108 | 22.15 ± .3711 | | | 0.16 ± 0.11/0.55 |
| {1} (d) [0 deg] | 9.06 ± .1433 | 22.39 ± .3883 | | | 0.19 ± 0.14/0.65 |
| {2} (a) [0 deg] | 8.57 ± .5596 | 28.84 ± .1770 | 8.95 | 26.87 | 0.17 ± 0.10/0.42 |
| {2} (a) [270 deg] | 9.01 ± .0561 | 26.10 ± .1315 | | | 0.12 ± 0.11/0.45 |
| {3} (a) [0 deg] | 7.99 ± .2755 | 26.65 ± .2105 | 8.00 | 24.58 | 0.19 ± 0.07/0.35 |
| {3} (a) [90 deg] | 9.62 ± .1132 | 25.58 ± .3166 | | | 0.11 ± 0.12/0.68 |

2-D Models, Estimated Geometry

| | | | | | |
|---|---|---|---|---|---|
| {1} (e) [0 deg] | 9.01 ± 0.10 | 26.65 ± 0.21 | 9.16 | 22.9 | 0.17 ± 0.13/0.70 |
| {1} (f) [0 deg] | 9.18 ± 0.10 | 22.42 ± 2.28 | | | 0.16 ± 0.11/0.63 |
| {3} (e) [0 deg] | 10.98 ± 0.47 | 24.24 ± 0.38 | 8.00 | 24.58 | 0.22 ± 0.17/0.81 |

3-D Models, Known Geometry

| | | | | | |
|---|---|---|---|---|---|
| {4} (a) [0 deg] | 11.02 ± 0.01 | 25.22 ± 0.05 | 11.37 | 23.25 | 0.10 ± 0.10/0.51 |
| {4} (a) [90 deg] | 11.02 ± 0.01 | 25.23 ± 0.06 | | | 0.14 ± 0.12/0.46 |

During the testing, a Siemens Sonoline Antares ultrasound system (Siemens Healthcare USA, Mountain View, Calif.) with a VF10-5 linear-array probe images the phantom at 8 MHz to observe internal structures. Those skilled in the art will recognize that other ultrasound systems or any imaging method that allows estimation of internal displacements may be employed without departing from a scope of one or more claims. Additionally, it may be possible to estimate internal displacements without any imaging. The testing scanner is configured with an Ultrasound Research Interface (URI) capable of recording radiofrequency (RF) echo data corresponding to an image region displayed on the monitor for off-line processing. To acquire data, the ultrasound system (US) probe face is positioned flush with the top surface of the phantom and in the x₁; x₃ plane that are, respectively, the lateral and elevational axes of the transducer. The bottom surface of the phantom is fixed to a rigid base so it does not slip as the probe compresses the top phantom surface. Other surfaces are free to move. Coupling gel is applied to the top phantom surface and approximates a free-slip boundary during compression.

Autoprogressive Algorithm: The Autoprogressive Algorithm is diagrammed in FIG. 2. AutoP uses the fact that equilibrium and compatibility are satisfied in any FEA; equilibrium relates applies forces 130 to stresses 140 and compatibility relates applied displacements 150 to strains 160. Since the measured forces 130 are applied, the computed stresses 140 in FEA 1 110 are closer to the actual stresses in the material. Similarly, application of measured displacements 150 in FEA 2 120 produces computed strains 160 closer to the actual strains in the material. These stresses and strains are collected and used to update the ANNs 170,180. As this iterative process continues the computed stresses 140, strains 160, and trained ANN material models 170,180 increasingly lead to more accurate representation of the tissue being examined.

ANNs are selected as the machine learning component of AutoP because of their inherent nonlinearity, robustness, and ability to learn complex material properties. Their highly parallel structure accumulates information as it becomes available experimentally. A general concern when using ANNs is determining the size of the network to be used. ANNs with too many nodes have a capacity that far exceeds what is necessary to learn mechanical behavior and can generate overfitting errors. On the other hand, if there are too few nodes the ANNs do not have enough capacity to store the information. Thus, it is preferable to implement adaptive ANNs to minimize these issues, although other methods of determining the number and/or size of ANNs may be employed without departing from a spirit and scope of one or more of the claims. Those skilled in the art will recognize that it may be possible to employ as few as a single ANN to model the entire object without having to know anything about the distribution of materials. Although the size of the ANN will still be determined for optimization purposes. During testing, feed-forward, fully connected neural networks with two hidden layers were employed. For more complex behaviors with additional positions as input to the ANNs, a nested architecture will most likely be employed. Sub networks that have stress and strain history points will feed one-way into the main network. Similarly, a subnetwork with the coordinates as inputs will feed one-way into the main network. Also, resilient propagation (RPROP) is employed to train the neural networks once the stress-strain pairs are obtained from FEA 1 and 2. Although those skilled in the art will recognize that this is a design choice and other methods of training the ANNs may be employed without departing from a spirit or scope of one of more claims. Once trained for the full applied load range, stress and strain vectors can be generated in AutoP. With data acquisition and model building complete, the model is now used to conduct numerical experiments within the range of training. During this last phase, elastic modulus and other parametric images are formed.

Autoprogressive pre-training: Before spatial information is considered, the ANNs are initialized using linear-elastic equations. This phase is for ANN initialization only. Given the nature of the phantom measurements, the ANNs are pre-trained for 2-D plane-stress conditions using the constitutive equation at the bottom of FIG. 3. A Poisson's ratio of 0.5 and a Young's modulus different from the measured value of the gelatin material is used to build unique constitutive matrices for the background and inclusion ANNs. One-hundred random strain vectors in a range expected experimentally are input to the equation 200 at the bottom of FIG. 3 and the resulting stress vectors are collected. Those skilled in the art will recognize that the number of strain vectors input is a design choice and fewer or more than 100 vectors may be input. The same strain vectors are input to the ANN 210 at the middle of FIG. 3 to generate stress vectors. The difference between the corresponding stress vectors result in 100 error vectors, each 3×1. ANN connection weights are adjusted by back propagating the error vectors through each network. This cycle is repeated a total of ten times for each ANN. Again this number is a design choice. At this time ANN initialization is complete and AutoP is ready for model building with experimental data.

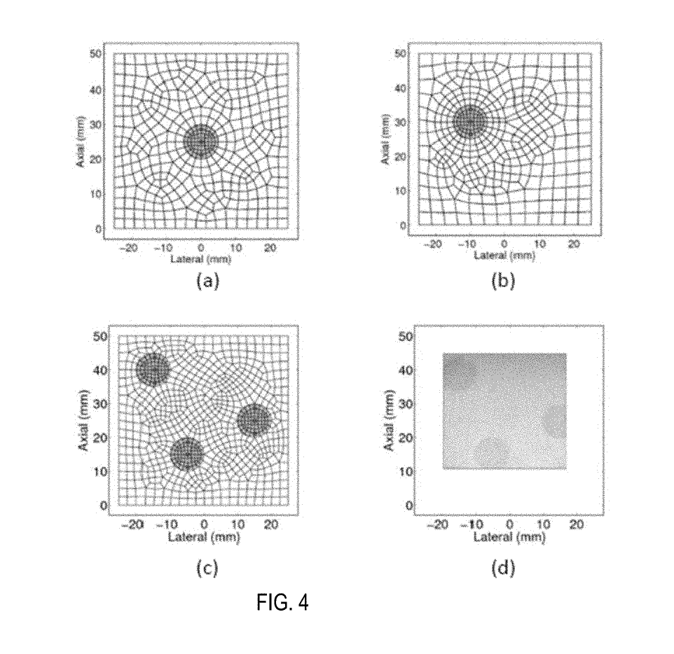

Autoprogressive training: The basic shape of the object and major internal components are found using segmented sonograms although those skilled in the art will recognize that segmenting is a design choice. In the proof of concept study, 2-D shapes are known to avoid contending with potential segmentation errors. The object shape is then meshed as shown for the three phantoms a, b, c in FIG. 4. In this non-limiting example, the first load step is a downward (x₂-axis) force applied by the ultrasound probe 10 along the beam axis. Applied force p and x₂-axis displacements u measured at the phantom surface and at select points within each inclusion (see FIG. 1) are entered into the finite-element algorithms FEA1 110 and FEA2 120 (FIG. 2). The finite-element algorithms 110, 120 compute three-component stress and strain vectors for that iteration that relate to each other through the neural networks, one ANN for each of the two materials in the phantoms 30. After completing an iteration, stress vectors from FEA1 110 are paired with corresponding strain vectors from FEA2 120. The pairs are sorted to the appropriate ANNs 170, 180 and used to update the connection weights. For example, if the inclusion is modeled using eight elements, the stress and strain vectors generated within those elements are used to train the ANN describing inclusion behavior. Each iteration ends with a calculation of the displacement error between nodal displacement estimates in FEA1 110 and those supplied for use in FEA2 120. Convergence is determined by comparing the mean and maximum error to given thresholds as described below.

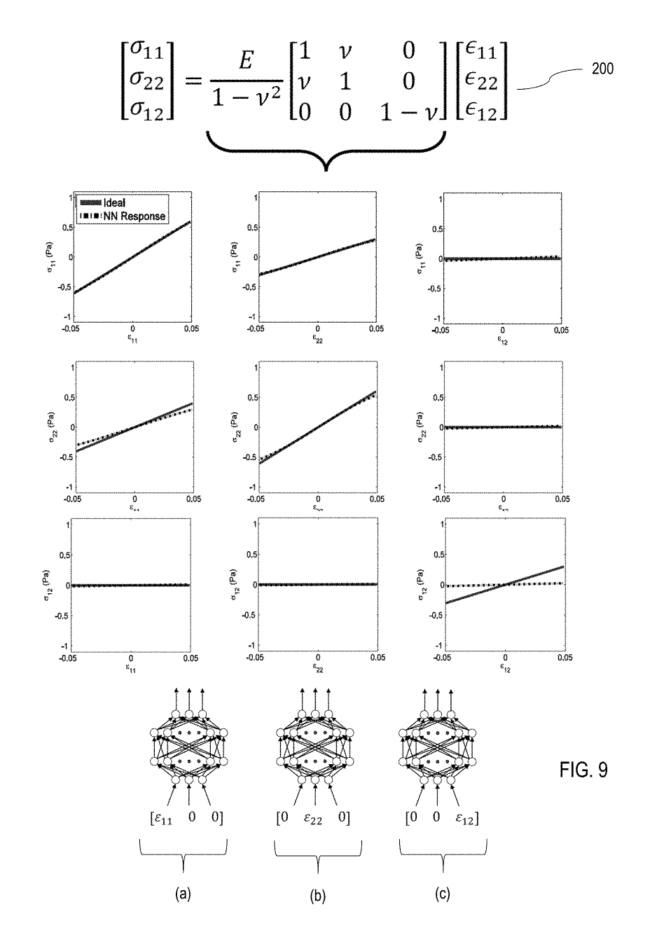

The above process is repeated for a preset number of iterations (in this example up to 5) and/or until specified convergence criteria are met. Training continues for each load step to complete a pass. An objective is to have FEA1 110 and FEA2 120 agree with each other and the measurements. Material properties of the medium are held nonparametrically in the ANNs, while approximations of spatial distributions of stresses and strains are computed in FEA1 110 and FEA2 120. The ANNs model the mechanical properties of the medium. Computational experiments (e.g., FEAs or other computational experiments such as the one described in FIG. 9, etc.) can be performed after training ANNs to estimate stress and strain distributions resulting from an applied force.

Convergence criteria: Convergence is determined during an AutoP training iteration by evaluating displacement errors 190. Errors defined as Δuᵢ=uᵢ−ûᵢ for the ith iteration are computed by comparing measured and modeled displacements as shown in.

FIG. 2. The first convergence criterion checks to see whether Δuᵢ/max(uᵢ)<0.5. The second criterion tests if mean(Δuᵢ)/max(uᵢ)<0.1, where the mean is taken over all nodes used in convergence checking. Threshold values are empirically determined. Those skilled in the art will recognize that the values 0.1 and 0.5 are design choices and other values could be selected without departing from a scope or spirit of one or more claims.

The variability of strain estimates is much greater than stress variability due to the use of internal displacement information in FEA 2 120. Therefore, the mean and standard deviation of strain values at meshed nodal points are calculated for each component material and used as follows: any stress-strain pair that has a component of strain fall outside of the mean±1 standard deviation is removed from the set of training data. Those skilled in the art will recognize that this is a mere design choice and need not be performed.

Displacement measurements are made at single points throughout the imaging plane for use in convergence testing that are not used in the FEAs. As discussed below, this practice helps AutoP learn about shear (x₁-x₂-axes) stresses and strains even though only axial (x₂-axis) displacements and surface forces are measured.

Effects of noise and measurement-point selection: FEA1 110 and FEA2 120 are independent of each other. Both use the ANN(s) in finding their respective solutions. They produce different nodal displacements when 1) the ANNs have not fully learned the mechanical behavior or 2) force displacement measurements contain noise.

Selecting nodes in the FEA mesh that receive displacement information helps keep these sources of modeling errors in check. In preliminary studies using ideal, noise free displacement information, including every node in the imaged region produce satisfactory results. The addition of measurement noise greatly affects the quality of the training data. When employing speckle tracking to estimate displacements, the resulting axial strains yield lower displacement error than lateral-displacement estimates because of the higher utility of phase information than amplitude information contained in echo signals. Significant measurement errors can be interpreted by AutoP as nonphysical force-displacement responses.

To minimize this occurrence, the force and displacement of the phantom surface under the rigid ultrasound probe 10 and several points within each inclusion are selected for the best results. Although this does not prohibit the selection of other points. Movements within stiff inclusions 80 generate the smallest axial displacement errors within the phantom 30. These few data are sufficient to accurately estimate the normal stress-strain vectors, but it takes more information to accurately estimate the shear components.

Experimental Design: AutoP combines FEA and ANN training methods with object shape information and force-displacement measurements to learn material properties of an object. The following phantom experiments were designed to explore the accuracy of informational modes developed using AuoP and discover methods for selecting free parameters such as convergence threshold values and the number and location of measurement points.

Four phantoms were used for investigations. The background materials in all Four had nominally the same stiffness and all the inclusions were stiffer than the background and equivalent to each other (Table I). Phantoms 2 and 3 have non-centrally positioned inclusions (FIGS. 4b and c) that were studied at several phantom orientations and probe locations as summarized in Tables I and II.

TABLE 1
Six different training scenarios for AutoP. These
are the cases referred to in Table 2 for Phantoms 1 and 3
(FIG. 5). Data set 1 are measurements for a centrally-located
US probe (FIG. 1). Data set 2 are measurements for an off-
center US probe location.
Cases for Phantom 1, Known Geometry

| | |
|---|---|
| Case (a) | training using data set 1 only |
| Case (b) | training using data set 2 only |
| Case (c) | training using data set 1 and |
| | then retraining on set 2 |
| Case (d) | training using both sets |
| | simultaneously |

Cases for Phantoms 1 and 3, Estimated Geometry

| | |
|---|---|
| Case (e) | Good segmentation - boundaries |
| | closely followed |
| Case (f) | Poor segmentation - large errors |
| | in boundary estimation |

The total probe displacement during compression was 1.5 mm, 3% of the 50 mm phantom height, which was applied in 30 equally-spaced steps. The total applied force ranged between 12-15 N. The total phantom deformation was kept small in these preliminary studies to ensure linear-elastic responses and avoid geometric nonlinearities. Those skilled in the art will recognize that these restrictions can be lifted. Axial force, probe position, and an RF echo frame are acquired after each probe displacement step. Internal displacements are estimated using a speckle-tracking algorithm applied to RF echo data.

AutoP is performed using equally-spaced load steps that span the entire range. Those skilled in the art will recognize that equally spaced load steps are a design choice and the steps may be unequal and still fall within a spirit or scope of one or more claims. For example, to train using 3 steps, input data from steps 1, 15, and 30, but to train using 10 steps, input data using steps 1, 3, 6 . . . 27, 30. Because of measurement noise, results are best when probe displacement and force estimates are included in AutoP training along with displacements at points in the inclusion and a few sparsely located throughout the background; background displacements are used for convergence testing as described below (although they may be used for training).

Informational models are better able to learn mechanical behavior when taught using diverse training data. In AutoP, one way to produce a richer set of training data is to apply a variety of spatial load distributions to the same phantom. For example, for phantom 1 4(a) data is acquired with the US probe centrally positioned in the x₁, x₃-plane (data set 1) and with the probe located 4 mm off-center along the x₁ axis (data set 2). From these two data sets on phantom 1 4(a), four training scenarios are explored as described as cases in Table II above.

Table I: Cases (a) and (b) compared with cases (c) and (d) explores how the amount of training data affects modulus measurement accuracy as reported for phantom 1 4(a) in Table II. Comparing results for cases (c) and (d) illustrates how the order of training data influences measurement accuracy. Cases (e) and (f) explore the effect of geometry estimation errors. The internal geometry of phantoms 1 and 3 was estimated and used in AutoP with the same data from case (a).

Results: FIG. 5 displays the reconstructed stress- and strain-vector maps for phantom 1 4(a) where trained AutoP informational models were used as material models during forward FEA processing. This group is referred to as the FEₐₙₙ results. Images 5(a)-(c) labeled axial, lateral, or shear stresses (top row) and 5(e)-(h) strains (bottom row) are relative to the orientation of the ultrasound beam along the vertical x₂ axis. The AutoP model is trained using surface force and displacement measurements and three internal displacement measurements made within inclusions.

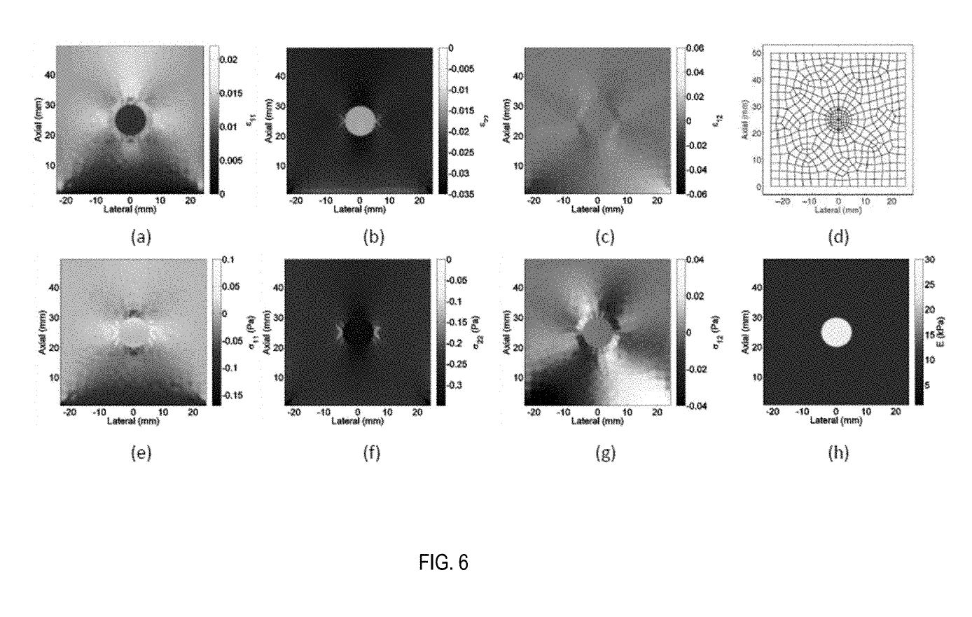

FIG. 6 contains FEA images corresponding one to one with those in FIG. 5. The material properties used to compute these results are obtained from indentation measurements, and are thus referred to as the FE_{ind} results. They are generated using conventional FEA forward modeling under the same loading and boundary conditions as those in FIG. 5. The background and inclusion elements assume a linear-elastic material model with Poisson's ratio v=0.5 and Young's modulus values E=9.16 kPa and 22.9 kPa, respectively.

Results of both figures are the output from FE analyses. The difference is that in FIG. 5 the element material properties were modeled by the trained ANNs of an AutoP model of phantom 1, whereas in FIG. 6 an ideal linear-elastic model was chosen using Young's modulus values estimated from indentation testing of phantom 1 materials. In both situations, the incompressible result v=0.5 and the equation

$$E ( {x}_{1} , {x}_{2} ) = \frac{( 1 - {v}^{2} ) {σ}_{22} ( {x}_{1} , {x}_{2} )}{v {ɛ}_{11} ( {x}_{1} , {x}_{2} ) + {ɛ}_{22} ( {x}_{1} , {x}_{2} )}$$

were employed to compute a Young's modulus, E(x₁, x₂), at each integration point in the mesh using data from the stress and strain maps. This resulted in the modulus images labeled (h) in both figures. Because axial forces and displacements were supplied to AutoP during training and the materials are incompressible, the axial and lateral stress and strain estimates are expected to be more accurate than shear estimates. Consequently, the second row of the above plane-stress equation from FIG. 3, is used to estimate E(x₁, x₂).

Imposing a nonslip condition at the base of the phantom and a free-slip condition at the top surface, provides variance in the stress and strain fields that do not show up in the modulus images. If they did show up in the modulus images, then the ANNs did not correctly learn the mechanical properties. Note that the experimental conditions for the data in FIG. 5 are not exactly the same as the ideal conditions assumed in the modeled results of FIG. 6. Thus, it is expected that the corresponding pairs of stress-strain maps will be similar but not an exact match.

Table II above lists the estimated values (based on the stress-strain response of ANNs trained with AutoP) and predicted values (from indentation) of Young's modulus for the three phantoms computed in the manner illustrated by FIG. 5. The same table contains results where ANNs were trained using different orientations of the phantoms and different data sets, labeled cases (a)-(d), and Young's modulus estimates when the internal geometry was estimated, labeled cases (e)-(f). The six cases are described in Table I above. Mean values measured from modulus images agreed with indentation estimates of moduli within 20%.

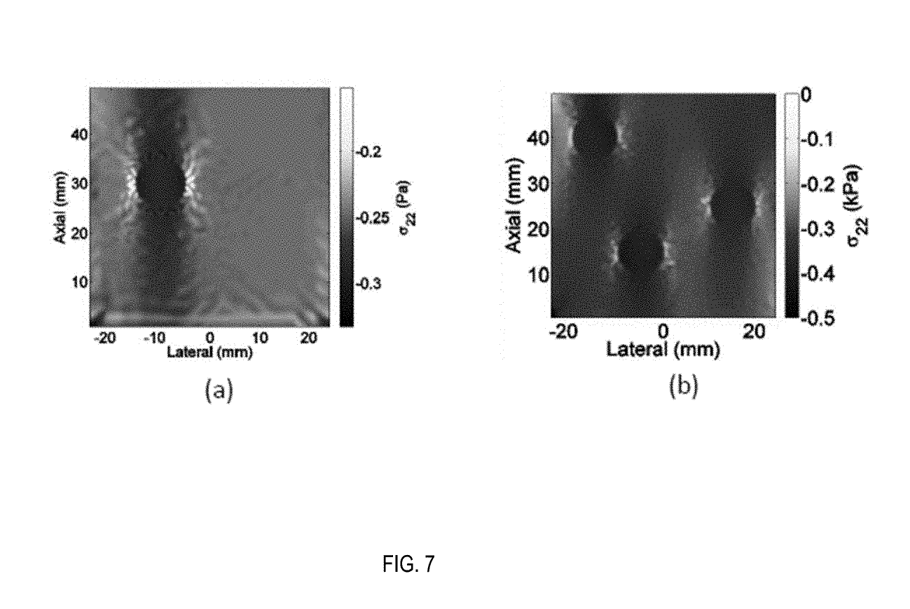

ANNs trained with AutoP have the ability to accurately estimate stresses and strains in an object. Comparing FIGS. 5 and 6 modest differences may be observed between measured and modeled normal stresses σ₁₁, σ₂₂ and normal strains ε₁₁, ε₂₂. The level of agreement suggests that measuring axial forces and displacements at the surface and within inclusions enables ANNs to learn to reliably predict normal stress and strains throughout a 5×5 cm² area with a spatial resolution on par with conventional FE modeling. As shown in FIG. 7 for σ₂₂, stress fields can also be measured for two slightly more complex phantom geometries.

The largest differences between measured and modeled stresses and strains involve shear components σ₁₂ and ε₁₂. Shear errors have little effect on modulus estimates only because shear vectors were not included in estimates of Young's modulus images via the above disclosed equation. Low shear accuracy indicates that more information needs to be provided during training with AutoP. This will be discussed further below.

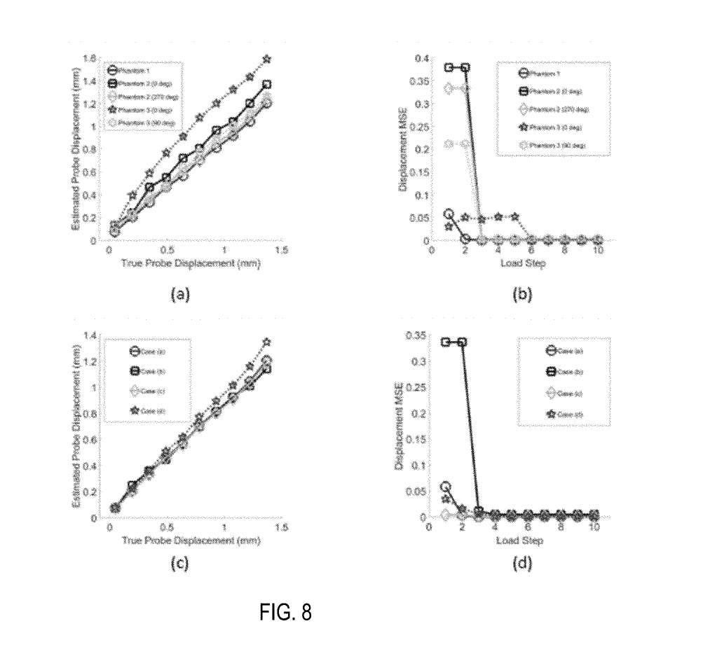

Quantitative Test 1: More quantitative tests of trained informational models can be found in FIG. 8. Because the position of the ultrasound probe in the motion controller is known with sub-micrometer accuracy, known and computed probe displacements are compared after each load step. During training, the probe is displaced and the resulting force is measured; conversely, in FEₐₙₙ that results in the stress and strain images of FIG. 5, forces are applied and displacements are calculated. Perfect mechanical characterization by ANNs would mean the measured and predicted values would match exactly. The results in FIG. 8a are for the three phantoms at two scanning angles. In FIG. 8b, the mean-squared error (MSE) between the displacements calculated in FE_{ind} and those estimated in FEₐₙₙ at the same locations in the imaged region are plotted as a function of load step for the same phantoms. Notice in FIG. 8b that following the first few load steps, AutoP converges suggesting it has learned what it needs from the available data. However, MSE remains large suggesting it has not learned the correct material properties yet. After the sixth load step, however, convergence results in an MSE for displacement that is less than 0.01 mm² for all phantoms studied. The data in FIGS. 8a and b are all for case (a), which trains ANNs using a single data set where the probe is centered on the top surface.

FIGS. 8c and d repeat the studies of 8a and b but only for phantom 1 where the data used during training with AutoP is varied among the different curves plotted. In all cases, adding low-noise data from a different view reduced the errors.

Quantitative Test 2 (Returning to the plane-stress equation expressed at the bottom of FIG. 3 and the top of FIG. 9): In FIG. 9, the constitutive matrix for these plane-stress conditions is multiplied by column vector [ε₁₁,0,0]^{T} where the strains ε₁₁ span a range slightly larger than that over which the ANN is trained. The stresses corresponding to these input strain are plotted in the first column 9(a) of plots in FIG. 9. The results are for phantom 1 case (d), which means the ANN is trained simultaneously on data acquired at two positions of the US probe on phantom 1. The solid lined curves in these plots are from the equation at the top (v=0.5), and the slopes of the lines correspond to matrix elements. Applying the same strain vector to the ANN material model yields the dotted curves in the same plots. When solid lined curves fall on dotted curves, the mathematical model and the ANN material model/informational model results agree. The second 9(b) and third 9(c) plot-columns in FIG. 9 are found by applying the vectors [0,ε₂₂,0]^{T} and [0,0,ε₁₂]^{T} respectively, to both the equation and the informational model. There is agreement between the results except for the plot of σ₁₂ versus ε₁₂ as seen qualitatively when FIGS. 5 and 6 are compared.

The center plot 9(b) of FIG. 9 displaying σ₂₂ versus ε₂₂, illustrates a consequence of using trained ANNs to predict stress values approaching or extending beyond the range of training data. There is a linear stress response for the strain range of training −0.03≦ε₂₂≦0.03, but the stress responses for larger input strain are nonlinear. The responses become linear when training is extended to the full range of testing.

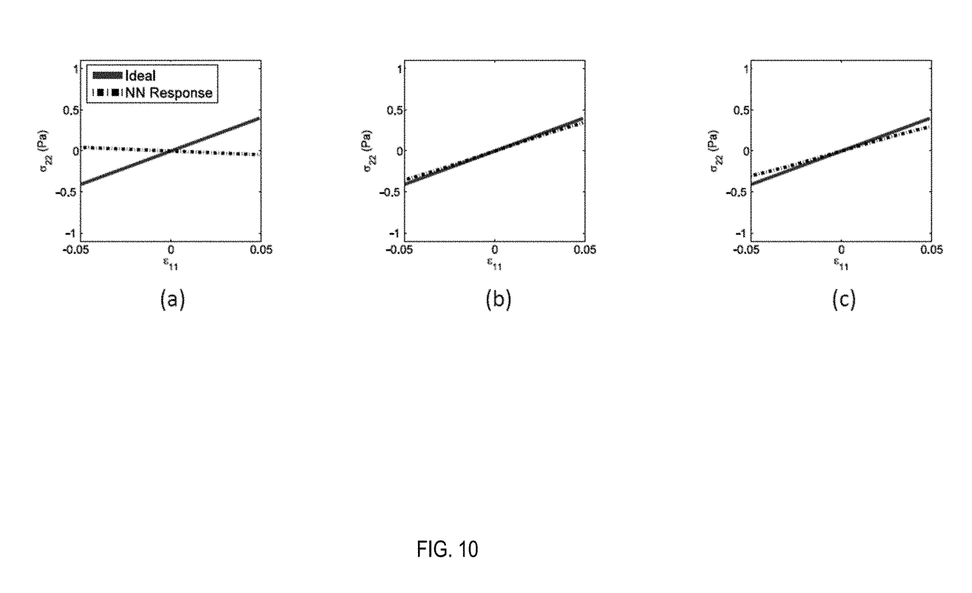

Varying the Amount and Uses of Training Data: FIG. 10 illustrates the influence on informational model accuracy of adding additional training data that convey material-property information. All three plots show the axial-stress responses σ₂₂ to isolated lateral strains ε₁₁ that are input to form different AutoP models of phantom 1. Plot (a) are the responses of an ANN trained with data from a single set, case (a) in Table I. The large disagreement between the solid lined and dotted curve data in FIG. 10a suggests not enough information was supplied to the ANN to learn the relationship between axial stress and lateral strain. Adding independent training data from another view (cases (c) and (d) in Table II) greatly improves the ANN's ability to characterize mechanical responses. Results of these informational models are shown in FIGS. 10b and 10c. However, there is a point at which adding more force-displacement data adds noise instead of new information. This is the point at which the informational model accuracy is reduced from over-training with noisy data.

The data in FIGS. 5, 6 and 9 illustrate that it is difficult to capture shear responses σ₁₂ and ε₁₂ by applying a uniaxial load while measuring forces and displacements only along the load axis. Although a shearing stimulus is not applied, there is a shear response because of the presence of inhomogeneities.

Several versions of AutoP training are performed to evaluate their effects on the learned shear response. These are: 1) training using axial force and displacement information (case (a) in Table I); 2) providing both axial and lateral displacement data during training; 3) imposing an assumption that the Jacobian matrix,

$$[ \frac{\partial σ}{\partial ɛ} ] ,$$

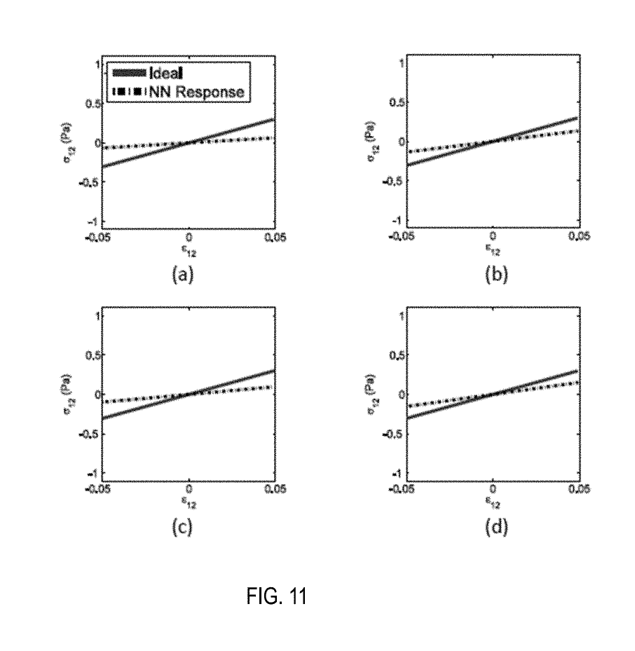

is symmetric during FE analysis; and 4) checking displacement errors for convergence at points not included in the training phase. The response of the AutoP model for training version (1)-(4) are plotted in FIG. 11 (a), (b), (c), (d) respectively.

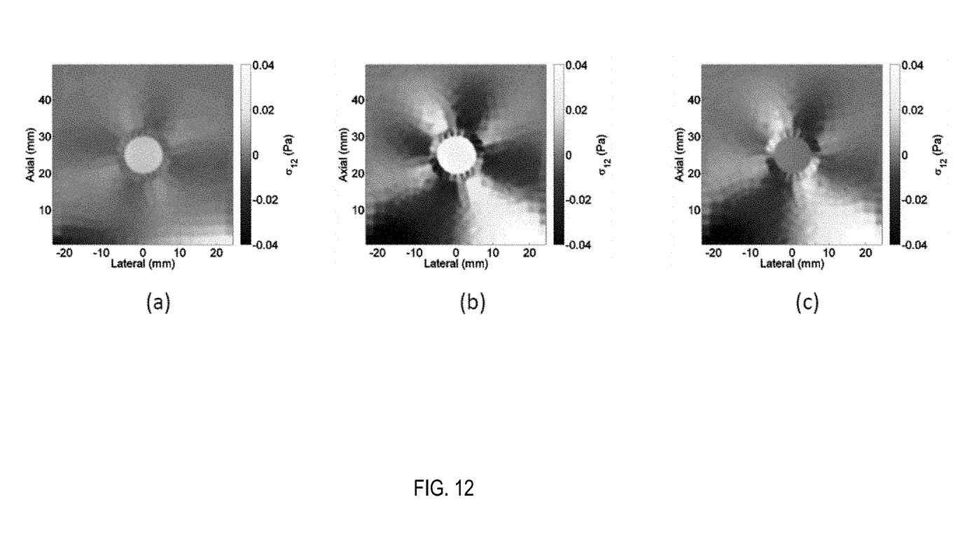

While none of these techniques leads to informational model agreement with the plane-stress model predictions of the shear stress-strain response, each of the adjusted training versions 2-4 improve the shear response compared to standard version 1. Improvements are indicated by an increase in the slope of the dotted curves closer to the solid lined curve. The greatest improvement occurs for version 4 when additional displacement data not included in FEA 2 120 are used in the check for convergence (FIG. 11d). A comparison of the full shear stress field is shown in FIG. 12. Shown are the estimated field with no training adjustment FIG. 12a, the version with stricter convergence criteria FIG. 12b, and ideal elastic behavior FIG. 12c. The similarity between FIGS. 12b and 12c suggest there is merit in including additional displacement data in the convergence check that are not provided in FEA 2 during training with AutoP.

The above results for the 2-D phantom experiments described under a uniaxial load and for plane-stress conditions show that the relevant stress and strain fields can be estimated with reasonable accuracy at a spatial resolution dictated by the FE mesh size. It is preferable, but not required, to select as few points as possible. Selecting 1-3 points for estimating axial displacements within each heterogeneity as seen in the B-mode image gives satisfactory results. These are added to measurements of force and displacement at the surface where the ultrasound probe contacts the object. Additional measurements that do not increase material property information but add noise may be counterproductive.

Step applications of the load and diversification of the scanning view while measuring displacements for nominally the same object locations improves informational model accuracy. Also, diversifying the locations of displacements estimated in the object, especially for convergence testing, allows the ANNs to learn the full range of stress and strain information. Interestingly, the training technique that works for phantom 1 and 2 having a single inclusion works equally well on phantom 3 with three inclusions, cases (e) and (f) of phantoms 1 and 3, and phantom 4, which is the 3-D version of phantom 1. Therefore, it appears that greater object heterogeneity does not significantly increase the training burden.

Informational models may be updated when new measurements are made. Coupling the ability of ANNs to learn new information with measurements taken at different times under different loading geometries, an existing ANN may be updated with the new data instead of building a new model. Case (c) of phantom 1 is an example of “retraining” previously developed informational models after new data is gathered. Providing new information to an existing informational model increases the accuracy of stress, strain, and Young's modulus estimates. This capability allows for resampling of a material after the initial measurements if it is found that the informational models are not converging or to initiate training of models for new materials under investigation.

Effects of Limited Information: Stress and strain vectors produced during uniaxial compression of cubic phantoms mostly span the axial and lateral spaces. Limited shearing occurs, resulting in less information available for training the ANNs on the shear behavior. However, it is possible for the ANNs to better learn the shear response by making adjustments to the training process. The most successful, but not the only available adjustment includes supplying more information during the displacement error calculation during the convergence check. Forcing the ANNs to more accurately predict displacements not given during training creates a more challenging convergence criteria, which in turn results in more training iterations. If the whole training process is thought of as reviewing flash cards, enforcing a stronger constraint during the convergence check is analogous to making more passes through the deck of cards.

When training involves the assumption of symmetry in the Jacobian matrix, there is no significant effect on the learned shear response. This may be largely due to the fact that displacements are given along a vertical line laterally centered within a symmetric model. Assuming a symmetric Jacobian matrix for phantom 1 would mostly effect the displacement symmetry within the FE model. Nodes being used in the convergence check lie on the line of symmetry, meaning virtually no extra constraint is being placed for convergence. It is possible that the symmetry assumption would have a larger effect on an asymmetric model or if nodal displacements were supplied asymmetrically throughout the mesh. However, the lack of improvement in ANN response with this assumption justifies its neglect.

Training Data Selection: Changing which nodes are given displacement information greatly affects the outcome of the informational modeling process. Three different cases illustrate this point, and in each case the only force measurements are those applied at the ultrasound probe. Consider situations where displacements are given 1) at the probe only, 2) along a vertical line through the inclusion, and 3) along a horizontal line through the inclusion. In all three cases, Young's modulus estimates of the background material are near the expected values; however, only in case (2) are accurate estimates for the Young's modulus of the inclusion achieved. The result of case (1) is not completely unexpected since the background makes up the bulk of the phantom and will have the greatest effect on the force-displacement relationship measured by the US probe. Results of (2) and (3) start to reveal what type of information needs to be supplied for training.

For each region in the material with distinct mechanical properties displacement information is given. For the models in this study, probe displacements would be enough to characterize the axial stress-strain relationship of the gelatin forming the background of the phantoms. Case (1) indicates that displacements be provided for the inclusion. The same number of nodes were given displacement data in cases (2) and (3), the only difference lies in the orientation of the nodes compared to the loading direction. In (2), the given information lies along a line parallel to the applied force whereas in (3) the nodes given displacement data spanned a line perpendicular to the loading direction. Because only axial displacement data is given, the movement of the nodes in (3) reveals very little information about how the points move relative to each other after loading—indeed, if the given displacements contained no noise, all of the nodes would have very similar (or identical) axial motion. However, in (2), the nodes compress relative to each other, producing more information about the material properties of the inclusion. The best sampling strategy for these plane-stress situations is empirically determined to be to measure displacements at nodes that vary significantly along the force gradient. For example, axial compression means the force gradient lies mostly along the x₂ axis. Therefore, nodes are selected along the x₂ axis in which displacements vary to the greatest extent.

Computational Load Considerations: During experimentation, training ANNs with AutoP initially took 102±14 minutes to complete on a quad-core processor operating at 3.4 GHz. With AutoP, most of the computation time was spent performing FEA 1 and 2. For two passes of ten load steps, a minimum of 60 FE analyses were performed, up to 220 in the worst-case where the maximum number of iterations was reached during each load step. Stress and strain image generation was on the order of minutes.

The training time for these 2D plane-stress models may be reduced by excluding the second pass, a heuristic could be included to monitor the displacement MSE and exit training if the error slope reduces to a predetermined value (although it is possible that this would terminate the training process before the full range of acquired data is presented—thus care would need to be taken if such criteria is implemented) or other methods may exist.

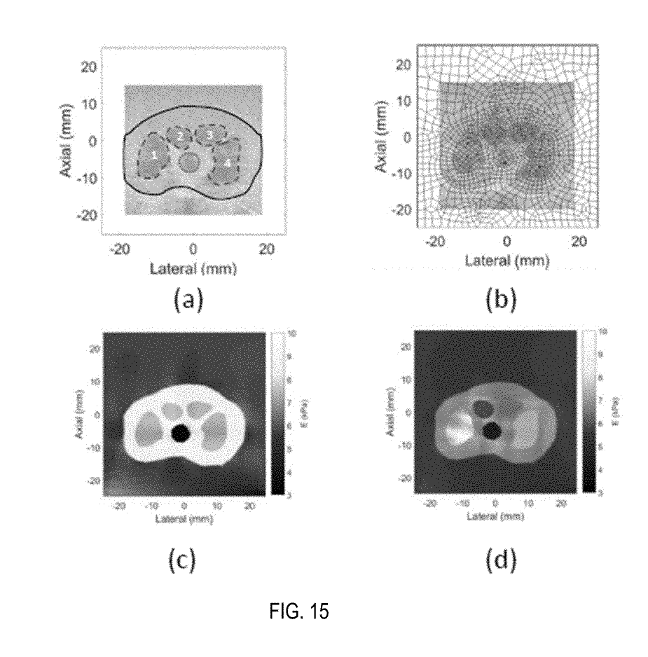

Rabbit Kidney Test: The ability of AutoP to build informational models of real biological tissues was tested using rabbit kidneys. Kidneys were excised from rabbits immediately post-mortem and embedded in a gelatin mixture similar to the background material in Phantom 1-4. An ultrasound B-mode image of the kidney-gelatin phantom is shown in FIG. 15a. The solid line denotes the boundary of the kidney, the dashed line indicates boundaries of the medullae, and the dotted line signifies the boundary of what may be part of the renal pelvis.

The kidney was manually segments and meshed (FIG. 15b) in the same manner and case (e) of Phantoms 1 and 3. Internal displacements estimated via speckle-tracking were provided in FEA 2 at the locations highlighted by dots in FIG. 15b.

In the first case of using AutoP to model the mechanical properties of this rabbit kidney, a total of four ANNs were used: one for the renal cortex (line region), one for all four medullae (dashed line), one for the region indicated by the dotted line, and one for the background gelatin material. After progressing through AutoP, the Young's modulus image displayed in FIG. 15c was generated. In that image, a clear contrast is seen between the different kidney regions and the background gelatin material. The background gelatin was estimated to have a Young's modulus value of 5.71±0.26 kPa where the value measure via indentation was ≈6 kPa.

When seven ANNs are used, instead of one ANN to characterize all four medullae, one ANN is used for each. Running through AutoP once more produces the Young's modulus image shown in FIG. 15d. The change in the Young's modulus for each medulla is likely due to the difference in orientation of the fibers to the applied load. The outer medullae have fibers oriented more orthogonal to the applied compressive load whereas the two inner medullae have fibers more parallel to the load. By allowing for a single ANN to characterize each region, anisotropy in the material properties of these rabbit kidneys is revealed. Table III lists the Young's modulus values estimated for each kidney model. Note for the seven ANN kidney model, the medulla are labeled 1-4 moving from left to right as seen in FIG. 15a. The results found in the seven ANN model compare well to those found in the literature (Gennisson, Jean-Luc, et al. “Supersonic shear wave elastography of in vivo pig kidney: influence of blood pressure, urinary pressure and tissue anisotropy.” Ultrasound in medicine & biology 38.9 (2012): 1559-1567).

TABLE III

| 4 ANN Kidney Model | | 7 ANN Kidney Model | |
|---|---|---|---|
| | Young's | | Young's |
| Region | Modulus (kPa) | Region | Modulus (kPa) |
| Renal Cortex | 8.62 ± 0.32 | Renal Cortex | 6.77 ± 0.19 |
| Renal Medullae | 7.32 ± 0.17 | Renal Medulla (1) | 7.88 ± 0.74 |
| | | Renal Medulla (2) | 5.86 ± 0.07 |
| | | Renal Medulla (3) | 6.37 ± 0.06 |
| | | Renal Medulla (4) | 7.27 ± 0.05 |
| Renal Pelvis | 3.2 ± 0.04 | Renal Pelvis | 5.36 ± 0.22 |
| Background | 5.71 ± 0.26 | Background Gelatin | 5.58 ± 0.11 |
| Gelatin | | | |

Having thus described preferred embodiments of the technology, advantages can be appreciated. Variations from the described embodiments exist without departing from the scope of the claimed technology. Informational models trained using AutoP have an ability to learn quasi-static mechanical behavior from measurements of surface forces and sparse displacement information. This approach has advantages when the mechanical properties of the medium are not well characterized, which is the situation in medical elasticity imaging. The conventional parametric inverse problem approach involving numerous assumptions is traded for a nonparametric machine-learning technique. This technique learns to model stress and strain fields from which imaging parameters are found. The ability of informational models to accurately predict mechanical behavior is limited by the type and amount of force-displacement information provided. Model accuracy can be improved by adding data from different spatial views and distributing measurement data for FEA training and convergence testing. Informational models have the ability to accumulate knowledge, which minimizes training time. Although particular embodiments and examples have been disclosed herein in detail, this has been done for purposes of illustration only, and is not intended to be limiting with respect to the scope of the claims, which follow. In particular, it is contemplated by the inventors that various substitutions, alterations, and modifications may be made without departing from the spirit and scope of the technology as defined by the claims. Other aspects, advantages, and modifications are considered to be within the scope of the following claims. The claims presented are representative of the technology disclosed herein. Other, unclaimed technology is also contemplated. The inventors reserve the right to pursue such technology in later claims.

Insofar as embodiments of the technology described above are implemented, at least in part, using a computer system, it will be appreciated that a computer program for implementing at least part of the described methods and/or the described systems is envisaged as an aspect of the technology. The computer system may be any suitable apparatus, system or device, electronic, optical, or a combination thereof. For example, the computer system may be a programmable data processing apparatus, a computer, a Digital Signal Processor, an optical computer or a microprocessor. The computer program may be embodied as source code and undergo compilation for implementation on a computer, or may be embodied as object code, for example.

It is accordingly intended that all matter contained in the above description or shown in the accompanying drawings be interpreted as illustrative rather than in a limiting sense. It is also to be understood that the following claims are intended to cover all of the generic and specific features of the technology as described herein, and all statements of the scope of the technology which, as a matter of language, might be said to fall there between.

Having described the technology, what is claimed as new and secured by Letters Patent is:

1. A computer-implemented method for estimating stress and strain distributions in biological tissue without requiring estimations of underlying constitutive models, the method comprising:
applying with a probe, a plurality of forces to the tissue;
recording a series of displacement measurements respectively over a period of time resulting from said plurality of forces; wherein said measurements are recorded from a plurality of locations on said tissue;
providing said force and displacement measurements to a processor based device; and,
using said processor based device to generate, based at least in part on the force and displacement measurements, said estimates of stress and strain distributions in said tissue.

2. The method according to claim 1 wherein said processor based device analyzes said stress and strain distributions to create a parametric summary of mechanical properties for said biological tissue.

3. The method according to claim 2 wherein said processor based device is programmed to implement the Autoprogressive (“AutoP”) Algorithm to create said parametric summary of mechanical properties for said biological tissue.

4. The method according to claim 3 wherein said AutoP Algorithm employs a plurality of Finite Element Analyses (FEAs) to respectively relate forces and stresses and displacement and strain.

5. The method according to claim 4 wherein said AutoP Algorithm relates said FEAs through at least one Artificial Neural Network (“ANN”).

6. The method according to claim 5 wherein said AutoP Algorithm relates said FEAs through a plurality of ANNs.

7. The method according to claim 6 wherein said plurality of ANNs are nested.

8. The method according to claim 6 wherein said plurality of ANNs area adaptive.

9. The method according to claim 1 wherein said plurality of forces generated by said probe are applied in stepped increments to the tissue, wherein said probe includes an ultrasound transducer which generates a radio frequency (RF) signal which is transmitted into the tissue.

10. The method according to claim 5 wherein said recording a series of displacement measurements includes recording an echo-signal frame and measuring a set of internal displacements.

11. The method according to claim 5 wherein said at least one ANN has at least one connected weight and further comprising training said at least one ANN by altering said at least one connected weight.

12. The method according to claim 1 wherein said series of displacement measurements are measured using a speckle-tracking algorithm.

13. A system for characterization of mechanical properties of biological tissue, the system comprising:
a force producing device;
a measuring device; wherein said measuring device is configured to measure external force applied to said biological tissue; and,
a processor in electrical communication with said measuring device; said processor programmed to measure a set of internal displacements from said tissue;
said processor being further programmed to develop an informational model of mechanical properties of the tissue using the Autoprogressive (“AutoP”) Algorithm.

14. The system according to claim 13 wherein said processor is further programmed to develop said informational model using a plurality of Finite Element Analyses (FEAs) to respectively relate forces and stresses and displacement and strain in said tissue.

15. The system according to claim 13 wherein said force producing device includes a probe, wherein the probe includes an ultrasound transducer which generates a radio frequency (RF) signal which is transmitted into the tissue.

16. The system according to claim 15 further including a positioning system connected to the probe configured to position the probe in relation to the tissue.

17. A method for generating a nonparametric, informational model of mechanical properties of biological tissue, the method comprising:
compressing, with an ultrasound probe having a linear array transducer, the tissue at a first position and transmitting a radio frequency (“RF”) signal into the tissue;
compressing with the ultrasound probe, the tissue at another position and transmitting the RF signal into the tissue;
acquiring and recording an RF echo frame each time said RF signal is transmitted into the tissue;
recording with a force-torque transducer, a surface force generated by the probe after each compression;
measuring an internal displacement using a speckle-tracking algorithm; and,
entering said force and said displacement into the Autoprogressive (“AutoP”) Algorithm.

18. The method according to claim 17 wherein said Autoprogressive Algorithm employs a plurality of finite element analyses (“FEAs”) to respectively relate said forces and a plurality of stresses and said displacements and a plurality of strains.

19. The method according to claim 18 further including relating the respective FEAs to each other through at least one artificial neural network (“ANN”); wherein said at least one ANN learns and records material properties by processing the force and displacement measurements.

20. The method according to claim 17 further including probing the informational model to find an imaging parameter without additional loading of the tissue.

21. The method according to claim 18 wherein said at least one ANN includes a plurality of connection weights and said at least one ANN is trained by updating said connection weights.
